(19) United States
(12) Patent Application Publication
(10) Pub. No.: US 2005/0172978 A1
(43) Pub. Date: Aug. 11, 2005

(54) Cuticle scrubber for human body

(76) Inventor: Tien-Jen Tien, Waipu Township (TW)

Correspondence Address: NIKOLAI & MERSEREAU, P.A., 900 SECOND AVENUE SOUTH, SUITE 820, MINNEAPOLIS, MN, 55402, US

(21) Appl. No.: 10/773,415

(22) Filed: Feb. 6, 2004

(51) Int. Cl. A61B 17/50

(52) U.S. Cl. 132/73

(57) ABSTRACT

A cuticle scrubber for human body includes a body made of flexible material so that the body can be transfigured to correspond to a grinding portion of the human body. The body has a bottom and a periphery coated with a glue layer surrounding the bottom. Multiple grinding particles equally scattered over and secured on the glue layer to form a grinding surface for grinding cuticle on the human body.

### BACKGROUND OF THE INVENTION

1. Field of the Invention

The present invention relates to scrubber, and more particularly to a cuticle scrubber for human body.

2. Description of Related Art

A conventional cuticle scrubber (1) in accordance with the prior art shown in FIGS. 1-4 comprises a scrubbing periphery (11) formed thereon. The conventional cuticle scrubber (1) includes multiple fine holes (111) defined therein and equally scattered on an outer periphery thereof to defined multiple dents for scrubbing cuticle on the human body or grinding nail. However, the conventional cuticle scrubber (1) may hurt the skin of the fingertip when grinding nail and the nail is inserted into the dent.

The convention cuticle scrubber (1) cannot be transfigured for corresponding to the curve of the grinding portion of the human body. Consequently, some grinding dead space is formed. For grinding the dead space with the conventional cuticle scrubber, the skin surrounding the dead space may be overly ground and hurt.

In addition, the conventional cuticle scrubber (1) is made of hard material. Consequently, the conventional cuticle scrubber (1) may be broken into pieces when falling to the ground or stricken by an object that is harder than the conventional cuticle scrubber (1). Furthermore, the conventional cuticle scrubber (1) is inconvenient for being carried because the shape of the conventional cuticle scrubber (1) cannot be transfigured.

The present invention has arisen to mitigate and/or obviate the disadvantages of the conventional cuticle scrubber (1).

### SUMMARY OF THE INVENTION

The main objective of the present invention is to provide an improved cuticle scrubber that is flexible for corresponding to the grinding portion of the human body.

To achieve the objective, the cuticle scrubber in accordance with the present invention comprises a body made of flexible material so that the body can be transfigured to correspond to a grinding portion of the human body. The body has a bottom and a periphery coated with a glue layer surrounding the bottom. Multiple grinding particles equally scattered over and secured on the glue layer to form a grinding surface for grinding cuticle on the human body.

Further benefits and advantages of the present invention will become apparent after a careful reading of the detailed description with appropriate reference to the accompanying drawings.

### BRIEF DESCRIPTION OF THE DRAWINGS

FIG. 1 is a perspective view of a conventional cuticle scrubber or human body in accordance with the prior art;

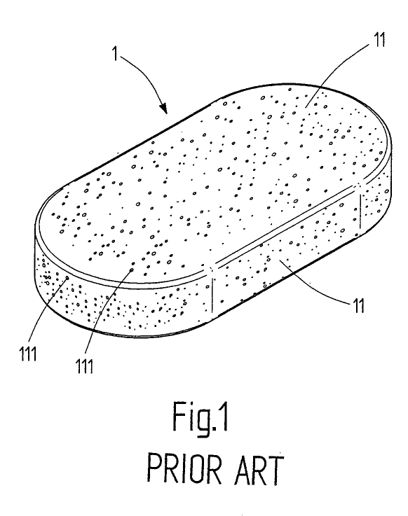

FIG. 2 is a partially perspective view of the conventional cuticle scrubber in FIG. 1;

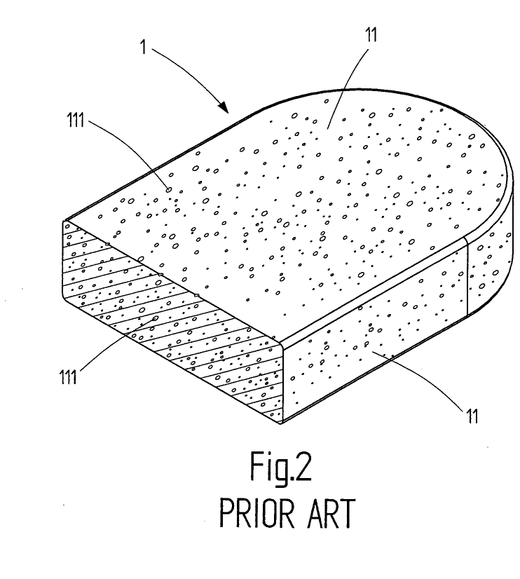

FIG. 3 is a first operational view of the conventional cuticle scrubber in FIG. 1;

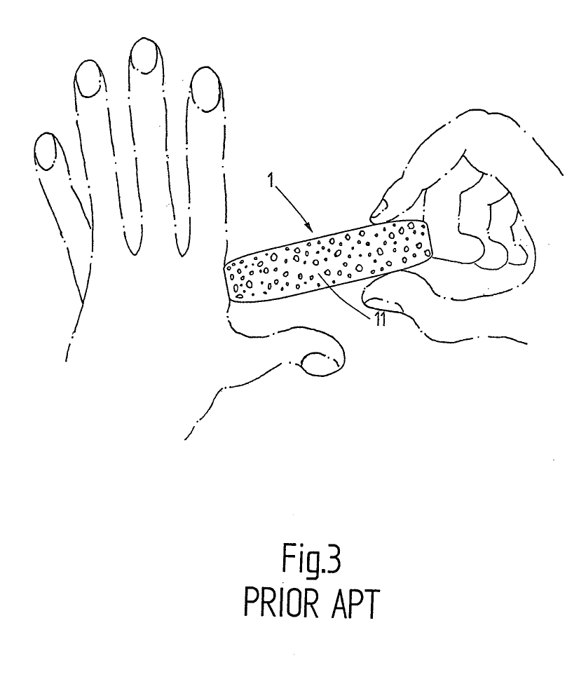

FIG. 4 is a second operational view of the conventional cuticle scrubber in FIG. 1;

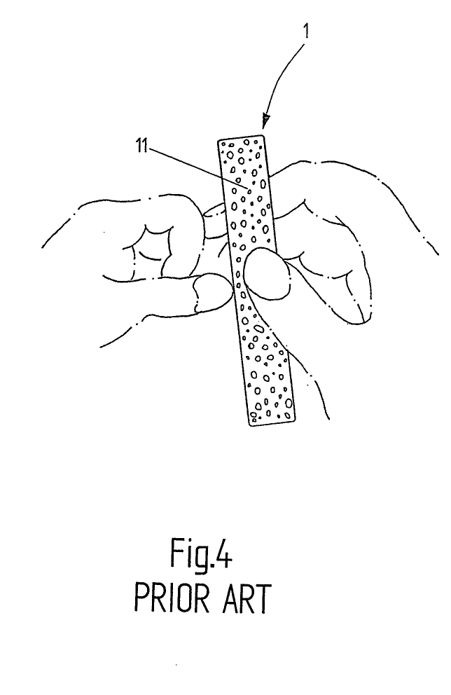

FIG. 5 is a perspective view of a cuticle scrubber for human body in accordance with the present invention;

FIG. 6 is a partially perspective view of the cuticle scrubber in FIG. 5;

FIG. 7 is a first operational view of the cuticle scrubber in FIG. 5;

FIG. 8 is a second operational view of the cuticle scrubber in FIG. 5; and

FIG. 9A is a perspective view of a second embodiment of the cuticle scrubber for human body in accordance with the present invention;

FIG. 9B is a perspective view of a third embodiment of the cuticle scrubber for human body in accordance with the present invention; and

FIG. 9C is a perspective view of a fourth embodiment of the cuticle scrubber for human body in accordance with the present invention.

### DETAILED DESCRIPTION OF THE INVENTION

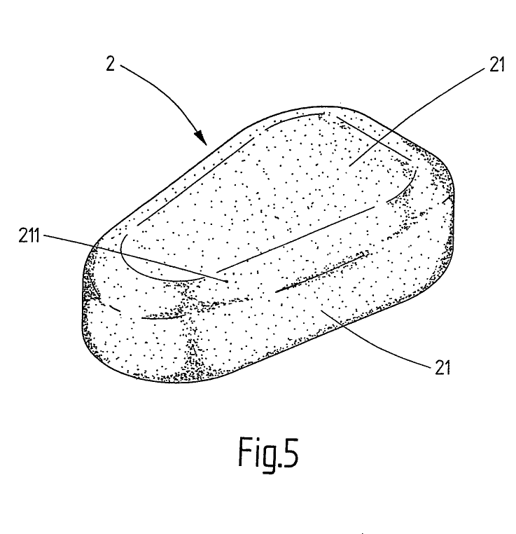

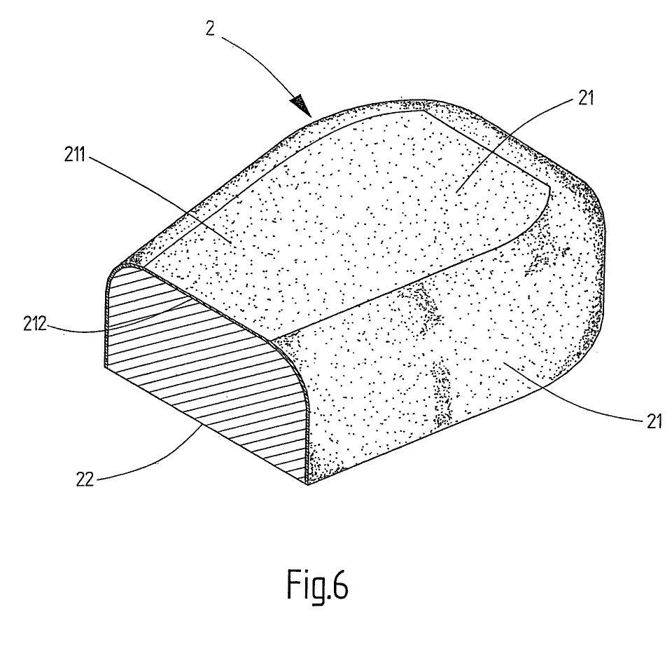

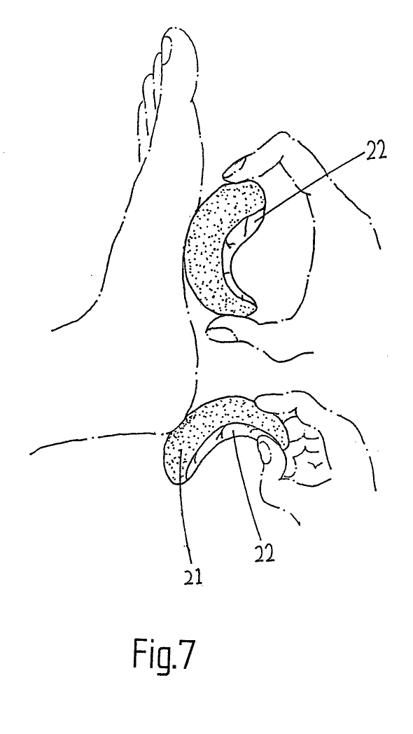

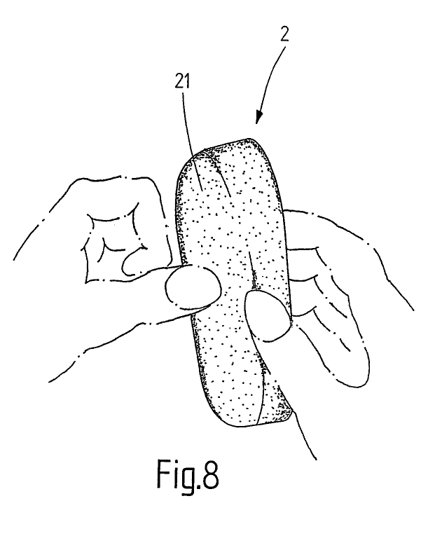

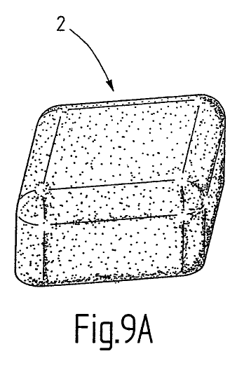

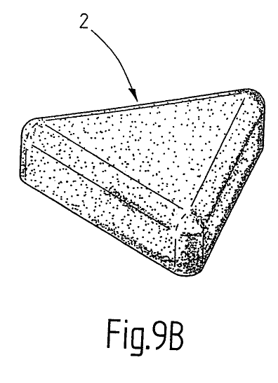

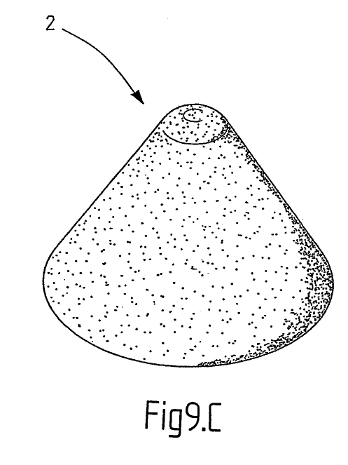

Referring to the drawings and initially to FIGS. 5-8, a cuticle scrubber for human body in accordance with the present invention comprises a body (2) made of flexible material. In the preferred embodiment of the present invention, the body (2) is made of ethylene vinyl acetate copolymer (EVA). Consequently, the body (2) can be transfigured to correspond to the grinding portion of the human body. As shown in FIG. 5, the body (2) is water-drop-like. Further with reference to FIGS. 9A to 9C, the body (2) can a prism, a cube or a cone. The body (2) has a bottom (22) and a periphery coated with a glue 10 layer (212) surrounding the bottom (22). Multiple grinding particles (211) are equally scattered over and secured on the glue layer (212) to form a grinding surface (21).

As described above, the cuticle scrubber for human body in accordance with the present invention includes several advantages as follow.

1. The grinding particles (211) on the grinding surface (21) do not contact with and hurt the skin of the fingertip during grinding nail when the body is curved toward the nail.

2. The body (2) is made of flexible material, such as EVA. Consequently, the cuticle scrubber of the present invention can be curved to corresponding to the shape of the grinding portion of the human body for effectively scrubbing the cuticle on the human body.

3. The body (2) of the present invention is flexible so that the body (2) does not be broken when falling to the ground or being stricken by a hard object. Furthermore, the body (2) can be folded to a shape that corresponds to the storing space. Consequently, the cuticle scrubber of the present invention can be conveniently carried.

Although the invention has been explained in relation to its preferred embodiment, it is to be understood that many other possible modifications and variations can be made without departing from the spirit and scope of the invention as hereinafter claimed.

1. A cuticle scrubber for human body, comprising:
a body made of flexible material so that the body can be transfigured to correspond to a grinding portion of the human body, the body having a bottom and a periphery coated with a glue layer surrounding the bottom and multiple grinding particles equally scattered over and secured on the glue layer to form a grinding surface for grinding cuticle on the human body.

2. The cuticle scrubber as claimed in claim 1, wherein the body is made of ethylene vinyl acetate copolymer (EVA).

3. The cuticle scrubber as claimed in claim 1, wherein the body (2) is water-drop-like.

4. The cuticle scrubber as claimed in claim 1, wherein the body is a prism.

5. The cuticle scrubber as claimed in claim 1, wherein the body is a cube

6. The cuticle scrubber as claimed in claim 1, wherein the body is a cone.
